(19) United States
(12) Patent Application Publication
(10) Pub. No.: US 2010/0041015 A1
(43) Pub. Date: Feb. 18, 2010

(54) COMPETITIVE ENZYME LINKED IMMUNOSORBENT ASSAY (C-ELISA) FOR THE DETECTION OF A FLAVIVIRUS SPECIFIC ANTIBODY

(76) Inventors: Bijon Kumarshi, Singapore (SG); Chenny Shi Cheng Li, Singapore (SG)

Correspondence Address: KNOBBE MARTENS OLSON & BEAR LLP, 2040 MAIN STREET, FOURTEENTH FLOOR, IRVINE, CA, 92614, US

(21) Appl. No.: 12/293,266

(22) PCT Filed: Mar. 16, 2007
(86) PCT No.: PCT/SG2007/000073

§ 371 Date: Jun. 9, 2009

(30) Foreign Application Priority Data
Mar. 16, 2006 (SG) 20061738-8

(51) Int. Cl. C12Q 1/70

(52) U.S. Cl. 435/5

(57) ABSTRACT

A competitive enzyme-linked immunosorbent assay (C-ELISA), using flavivirus member specific immunological agents was developed to detect antibody specific to members of the flaviviruses indicative of exposure to flavivirus. The test is based on a competition for epitope binding on the envelope protein of the flavivirus antigen captured using anti-flavivirus IgA in the presence of flavivirus positive serum. This test has comparable sensitivity specificity and speed to the virus neutralization assay (VNT). C-ELISA is a versatile technique, which could have various applications. Slight modifications of this protocol could lead to a C-ELISA-based detection method of secondary infection or one that could be used for serotype specific sero-epidemiological studies and/or vaccine evaluation. The protocol developed for C-ELISA was demonstrated using dengue lysate antigen and dengue specific monoclonal antibody. This can be used against other flaviviruses and the results for Japanese encephalitis illustrates this.

### FIELD OF THE INVENTION

The present invention relates specifically to a technique of detecting exposure of a human and/or an animal to a flavivirus or equivalent thereof. More particularly, the present invention relates to a rapid and easy analysis of a biological sample taken from a subject (animal or human) in order to determine if the subject had been exposed previously to a member of the flavivirus family or equivalent thereof. The present invention further provides the serotyping of flavivirus exposure based on serum analysis and provides medical diagnostic kits and differential sero-evaluation to members of a flavivirus infection or equivalent thereof. The invention is particularly important for the detection of flavivirus secondary infection, defining the serotyping of previous infection, sero-epidemiology and vaccine evaluation.

### BACKGROUND

The Flaviviridae family contains a myriad of viruses that cause disease in humans and are generally transmitted by mosquitoes and ticks. The Flavivirus genus contains a number of viruses including yellow fever virus (YF), dengue fever virus West-Nile (WN) and Japanese encephalitis (JE) virus which are responsible for their corresponding diseases.

Dengue is the most common and widespread arthropod-borne flavivirus infection in the world and more than 1.2 million dengue infections occur annually while 2.5 billion people are at risk living in the dengue endemic area of the world (WHO/TDR, April 2005). There are four distinct virus serotypes, each capable of producing a wide spectrum of signs and symptoms and a primary infection with one of the four serotypes confers lasting immunity to that type. The chance of secondary infection with different serotypes still remains.

Although dengue is endemic in most of the tropical and subtropical countries, it occurs mainly in developing countries that lack the healthcare infrastructure or financial capabilities to readily diagnose dengue effectively. In Singapore, an uneven distribution of the two vectors resulted in the uneven transmission of dengue within the population (Ooi et. al., 2001). By doing a serological survey, it was determined that a change in the location where dengue was acquired could have occurred. This finding may aid in the modification of vector control programs to target new areas of transmission. However, only four sero-epidemiological surveys have been completed so far. The high cost of laboratory tests or the lack of reagents needed contributes to so few surveys being conducted.

Traditionally, hemagglutination inhibition (HAI) was used to detect antibodies against dengue. However this method requires mouse brain-derived antigens that are not readily available due to the difficulties in growing such cells. Another method commonly used is complement fixation but this requires antibodies that are short-lived. More and more laboratories are resorting to the commercial enzyme-linked immunosorbent assay (ELISA) kits. However, none of these tests are specific to dengue antibodies and cross-react with other members of Flaviviruses co-circulating in the same region, like Japanese Encephalitis (JE), West Nile, Hepatitis C, Yellow Fever, Murray Valley encephalitis etc.

The sensitivities of the performance of six commercially available immunoassay systems for the detection of dengue virus specific immunoglobulin M (IgM) and IgG antibodies in serum (Goren et al. 2000) have recently been evaluated. The sensitivities of the dengue virus-specific assays evaluated varied between 71 and 100% for IgM and between 52 and 100% for IgG, with specification of 86 to 96% and 81 to 100% respectively. The total test time of these tests varied from 7 to 480 minutes. In addition to this, all tests require laboratory equipment support except Pan-bio RIT and its sensitivity and specificity for dengue IgG assay were very low (75% and 52% respectively). However, none of the tests evaluated in these studies were able to detect dengue specific antibody, but rather were cross-reactive with other flaviviruses. Hence, the detection of an early secondary infection is rather difficult at present, because only the presence of dengue specific IgG during an acute phase (febrile) of infection indicates secondary dengue infection (Schilling et al., 2004).

Sero-epidemiological studies have shown that secondary infection is a major risk factor for dengue haemorrhagic fever and dengue shock syndrome through antibody dependent syndrome enhancement (ADE) (Halstead et al., 1973, Halstead S. B., 1988). For epidemiological and pathological investigations, it is important to differentiate between primary and secondary virus infection and to determine dengue serotypes of past and current infections.

The most widely used dengue diagnostic technique is serological analysis of the suspected samples. However the technique is rather complicated because of several reasons—(i) patients may have multiple and sequential infections with the four dengue virus serotypes due to a lack of cross-protective neutralization antibodies; (ii) multiple and sequential flavivirus infections make differential diagnosis difficult due to the presence of pre-existing antibodies and original antigenic sin(many B-cell clones responding to the first flavivirus infection are re-stimulated to synthesize early antibody with a greater affinity for the first infecting virus than for the present infecting virus in every subsequent flavivirus infection) in regions where two or more flaviviruses are co-circulating; (iii) IgG antibodies have high degrees of cross-reactivity to homologous and heterologous flavivirus antigens; and (iv) the serodiagnosis of past, recent, and present dengue virus infections is difficult due to the long persistence of IgG antibodies (≧10 months, as measured by E/M-specific capture IgG ELISA, or life long, as measured by E/M antigen-coated indirect IgG ELISA) in many dengue patients with secondary infections (Gubler, D. J. 1996 and Innis et al 1989).

Thus, among the viral infections that can be diagnosed by serology, dengue virus infection is among the most challenging. However, great advances in analyzing the complex viral antigens and antibody responses have recently been made by the development of various methods that target different structural and non-structural (NS) proteins for serodiagnosis and seroepidemiological studies of dengue virus infection.

The haemagglutination inhibition (HAI) test has traditionally been used for the differentiation of primary and secondary dengue infection. It is less popular now due to the inherent disadvantages of the test (Innis et al., 1989 and Shu et al., 2003). In contrast, capture IgM and IgG ELISAs have become the most powerful assays for the differentiation of primary and secondary infection due to high sensitivity and specificity (Innis et al., 1989, 1997). However, for the serotyping of dengue infection particularly for the sero-epidemiological investigation, neither capture IgM ELISA nor IgG ELISA is useful because of cross-neutralizing antibodies among antibodies of dengue serotypes.

The virus neutralization test (VNT) is the gold standard technique currently available that can be used to distinguish between dengue and other flaviviruses (WHO manual 2002). The VNT is also used to discriminate among dengue serotypes (1-4 serotypes). Serum antibodies raised against either virus may neutralize all dengue serotypes and even other flaviviruses as well but would neutralize the homologous virus at a higher titre that it would neutralize the heterologous virus (Makino et al., 1994). Unfortunately, anti-dengue antibodies (IgM) developed at an early phase of infection give rises to an equal level of neutralizing against all dengue serotypes; hence secondary infection cannot be determined at this stage by VNT (Nawa et al., 2000). On the other hand, VNT has the following disadvantages:
i. Needs sophisticated cell-culture laboratory and trained personnel
ii. Duration of test is more than a week
iii. Serum from a patient with a dengue infection less than six months cannot be serotyped due to dengue cross-reactive antibodies
iv. Difficult to detect multiple dengue serotypes
v. 10% sera have toxic effect on cell culture
   Innis et al. (1989), first proposed classification of primary and secondary infections by determining the ratio of the units of dengue virus IgM antibodies to the units of dengue virus IgG antibodies. They showed that the acute-phase sera of patients with primary dengue virus infections had higher IgM/IgG ratios, whereas patients with secondary infections had lower IgM/IgG ratios. This method has made a great contribution to the analysis of the immune status of patients with dengue. Very recently, Shu et al., 2003, has modified and simplified the above method to differentiate the primary and secondary dengue virus infection using the ratio of IgM/IgG readings directly (>1.2 indicates primary infection or <1.2 indicates secondary infection, respectively) without calculating the antibody units through the use of a standard control.

Recently, Ludoffs et al., 2002 reported the serological differentiation of infections with dengue serotypes 1 to 4 by using recombinant antigens. Immunoblot strips dotted with the B domains of dengue virus serotypes 1 to 4 expressed in Escherichia coli used to detect dengue serotype specific antibodies in paired serum samples from 41 patients with primary and secondary infections. Although some cross-reactivity with heterologous serotypes were observed, the results showed 93.94% specificity, however serotyping is not reliable for secondary infection by this method.

The problems faced for dengue virus is equally encountered by other flaviviruses, which share antigenic characteristics, hence make difficult in accurate diagnosis. There is a need, therefore, for a rapid, reliable, and simple test that can distinguish flaviviruses specific antibody at any stage of infection from antibody to other flaviviruses as well as among flavivirus serotypes. Serological detection procedures have the potential role to play in clinical diagnosis to differentiate flavivirus infections from other infectious diseases similar to flavivirus as well as in sero-surveillance studies and vaccine valuation. Such techniques will assist in the early detection of secondary infection leading to better case management, reduced case fatality and cost of treatment.

### SUMMARY OF THE INVENTION

In a first aspect of the present invention, there is provided a method for detecting exposure of a subject to a flavivirus or equivalent thereof, said method comprising:
contacting a biological sample from the subject with a mixture of anti-flavivirus IgA captured components of flavivirus or an equivalent;
subjecting the biological sample and the anti-flavivirus IgA captured components to a competing flavivirus specific immunological agent;
determining the presence of a complex that forms between a flavivirus specific binding partner present in the biological sample and an anti-flavivirus IgA captured component in the presence of the competing flavivirus specific immunological agent.

The present invention results from a need to develop a rapid, low cost and straightforward assay to determine present or prior specific exposure to a flavivirus. The invention specifically utilizes a competing flavivirus specific immunological agent to compete with a binding partner from the host body for the epitope either specific to flavivirus or to their serotypes presence on the envelope protein of flavivirus, which includes a mixture of anti-flavivirus IgA captured components including flavivirus particles amongst other antigens indicative of flavivirus infection.

Accordingly, the present invention shows greater specificity and sensitivity compared to other conventional and most currently used flavivirus IgG indirect or capture ELISA by providing a platform that can specifically identify antibody (IgG) produced against flavivirus or immunological relatives thereof at any stage of the infection.

Preferably, the flavivirus is selected from a group including yellow fever virus, dengue fever virus, JE virus, West Nile virus, Hepatitis C virus, Murray Valley virus and St. Louis encephalitis.

In another aspect of the invention there is provided a kit for detecting exposure of a subject to a flavivirus or equivalent thereof, said kit comprising;
anti-flavivirus IgA captured components from flavivirus;
competing flavivirus specific immunological agent; and
at least one detection agent for detecting a complex that forms between a binding partner present in a biological sample and an anti-flavivirus IgA captured component in the presence of the competing flavivirus specific immunological agent.

The present invention also provides individual components of the kit for use in the method of the present invention including solid supports such as microtitre plates and nitrocellulose membranes on which the anti-flavivirus IgA captured components are immobilized.

The present invention also provides a method of assessing the relative risk of one or more subjects being exposed to flavivirus or its equivalent thereof within a defined location (e.g. geographical area, housing estate, means of transport or center for medical treatment or assessment), comprising;
a) obtaining samples from a representative population within a defined location; and
b) assessing evidence of exposure of individual members of a sample population to a flavivirus or equivalent thereof by the method comprising the steps of—
   i) contacting a biological sample from subject with a component derived from the flavivirus or equivalent thereof to form a complex between the components and a subject-derived binding partner of the component contained within the biological sample;
   ii) determining the presence of the complex, wherein the presence of the complex is indicative of exposure of the subject to a flavivirus or equivalent thereof; and
c) assessing the relative risk of exposure within the defined location.

Risk analysis may be conducted using software in a computer readable form. Consequently, the present invention further relates to a computer readable program and computer comprising suitable for analysing exposure of subjects or group of subjects or a risk of exposure of subject or group of subjects to a flavivirus or equivalent thereof.

### FIGURES

FIG. 1 shows the optimization of dengue lysate antigen on the anti-dengue IgA captured polystyrene plate. FIG. 1 (A) shows a comparison of MAb-captured and anti-dengue IgA captured ELISA. (a) Detection of dengue virus (serotype-2) using cell supernatant virus antigen. Virus was diluted at 10-fold dilution (Log₁₀) starting 10⁶ pfu/ml to 10² pfu/ml in virus diluent. (b) Detection of dengue virus (serotype-2) using cell lysate antigen. In both cases anti-dengue IgA captured ELISA showed better performance than MAb-captured ELISA.

FIG. 2 shows the optimization of Monoclonal antibodies against each serotype of dengue lysate antigens. Determination of the optimal dilution of MAbs. (a). Serial two-fold dilutions of each MAb (1:250 to 1:32000) were reacted with 2-fold dilution of dengue positive and two dengue negative sera (one was negative to dengue indirect IgG ELISA and other one was positive to IgG ELISA and yellow fever VNT but negative to dengue VNT) in a C-ELISA. A MAb dilution of 1:2000 was chosen.

FIG. 3 shows optimization of dengue IgG positive and negative sera using competitive ELISA (C-ELISA). Determination of an optimal dilution of serum for C-ELISA. Serial two fold dilutions of negative, flavi-cross-reactive and positive control sera were reacted with the optimal pan-den MAb dilution (1:2000) in a C-ELISA. To minimize the volume of serum required, a serum dilution of 1:20 was chosen as the preferred dilutions that performed only slightly better.

FIG. 4 shows the determination of cut-off point of C-ELISA using VNT confirmed dengue negative and positive sera. Establishment of a negative cutoff point for the C-ELISA. (a) All dengue IgG negative sera (n=100); (b) Dengue IgG positive sera detected by VNT (n=64). (b) The dotted line represents the cut-off point of 30% inhibition (mean plus 3 standard deviation) for distinguishing between negative and positive samples.

FIG. 5 shows the comparative evaluation of three dengue specific IgG detecting techniques, blocking ELISA, simultaneous and pre-incubated competitive ELISA. Comparison of B-ELISA with C-ELISA (Simultaneous and Pre-incubation addition of MAb). Each datum point is the mean of four values; moderate positive (Log₂ 100 TCID₅₀, 8) and negative sera. The simultaneous addition of test serum and monoclonal antibody showed low level of performance (positive 1:40) compared to pre-incubation and blocking ELISAs (1:160).

FIG. 6 shows the results of C-ELISA using 10 triplets serum samples (collected on day-1, day-4 and day-20) from dengue confirmed cases. The presence of dengue specific IgG was detected from the triplet samples from dengue confirmed 10 patients (by PCR) at day 1, day 4 and day 20. The levels of anti-dengue specific IgG in the second and third collections were increased substantially compared to first collection.

FIG. 7 shows results of correlation coefficient between C-ELISA and VNT. Linear regression of anti-dengue IgG specific serum samples shows a high level of correlation between C-ELISA and VNT (r=0.91) and indicates a high level of specificity compare to “gold standard” technique.

FIG. 8 shows the correlation coefficient between 30 and 15 minutes one step C-ELISA. Linear regression of two assays shows high level of correlation (r=0.9675).

### DETAILED DESCRIPTION OF THE INVENTION

In a first aspect of the present invention, there is provided a method for detecting exposure of a subject to a flavivirus or equivalent thereof, said method comprising:
contacting a biological sample from the subject with a mixture of anti-flavivirus IgA captured flavivirus viral components of flavivirus or an equivalent;
subjecting the biological sample and the anti-flavivirus IgA captured components to a competing flavivirus specific immunological agent;
determining the presence of a complex that forms between a flavivirus specific binding partner present in the biological sample and an anti-flavivirus IgA captured component of the flavivirus in the presence of the competing flavivirus specific immunological agent.

The present invention results from a need to develop a rapid, specific, low cost, straightforward assay to determine present or prior exposure to a flavivirus. The method uses anti-flavivirus IgA to capture flavivirus antigen preferably from crude cell lysate. The lysate derives from cells infected with flavivirus comprising a mixture of flavivirus components, preferably immunogenic components. Subjects including animals such as mammals and in particular humans are screened for the presence of flavivirus specific binding partners in a biological sample in the presence of a competing flavivirus specific immunological agent. The preferred binding partners are subject-derived binding partners such as, but not limited to immunointeractive molecules. Preferably, the immunointeractive molecules are antibodies particularly immunoglobulin G (IgG) or immunointeractive fragments. Furthermore, specificity is enhanced with the use of a flavivirus specific immunological agent as a competitor of the flavivirus specific binding partner in the biological sample. The identification of such binding partners or competing flavivirus specific immunological agents is then used as evidence of present or prior exposure of the subject to flavivirus or an equivalent thereof by their ability to form a complex with the anti-flavivirus IgA captured component. Hence the invention utilizes a combination of anti-flavivirus IgA captured components and C-ELISA.

Accordingly, the present invention shows greater sensitivity and specificity compared to other conventional and most currently used flavivirus IgG ELISA by providing a platform that can identify antibodies produced against flavivirus or immunological relatives thereof at any stage of the infection.

Throughout the description and claims of this specification, use of the word “comprise” and variations of the word, such as “comprising” and “comprises”, is not intended to exclude other additives, components, integers or steps.

### Flaviviruses

The term “flaviviruses” or “flavivirus” as used herein the claims and description includes the Flaviviridae family of flaviviruses including the flavivirus genus which cause disease in humans and is generally transmitted by arthropods such as mosquitoes and ticks. The viruses are responsible for diseases such as but not limited to yellow fever, dengue fever and JE. The species of flaviviruses which make up the genus have demonstrated some conservation of sequences at the nucleotide and amino acid sequence level. The viruses included in the genus of flaviviruses include but are not limited to yellow fever virus, dengue virus West Nile and JE virus. Due to similarities at the nucleotide and amino acid level, these viruses may show similarities in antigenicity, transmission and disease.

### Dengue Virus

The term “dengue virus” as used herein the claims and description refers to all dengue serotypes (Den-1, Den-2, Den-3 and Den-4) associated with a dengue infection. The present invention is applicable to detecting dengue virus infection or exposure in any subjects including human, non-human animals and laboratory animals. Human subjects, however, are preferred in accordance with the present invention. However, the invention includes any subject that can respond to an infection or immunization by the dengue virus or an equivalent thereof.

A dengue virus is defined as a group of RNA human virus consisting of enveloped particles of about 40-50 nm in diameter. The viral genome is approximately 11 kb (Stollar et al, 1966). Mature virion consists of a positive sense RNA genome enclosed by an isometric nucleocapsid. The genome encodes a single open reading frame of about 11000 nucleotides, coding for the three structural (C-Capsid, M-Membrane and E-Envelope) and seven non-structural (NS1, NS2a and NS2b, NS3, NS4a and NS4b, NS5) proteins.

Dengue virus is transmitted to humans through the bites of infected female Aedes mosquitoes, principally the A. aegypti mosquito. This is a small, black and white, highly domesticated tropical mosquito that prefers to lay its eggs in artificial containers found in and around homes that may hold water such as buckets, flower cases and other water containers. The adult mosquitoes are rarely noticed outside; they usually rest in dark indoor sites, are unobtrusive and prefer to feed on humans or animals during the day light hours, with most biting activity occurring in the early morning or late afternoon (Gubler et al., 1992; Newton et al., 1992). The female mosquitoes are nervous feeders, disrupting the feeding process at the slightest movement of the host, thus returning to the same or different host to continue feeding. Because of this behaviour the mosquito often feeds on several persons during a single blood meal and if infective may transmit the virus to multiple persons (Platt et al, 1997; Scott et al., 1997). Such behaviour has been used to explain the epidemiological observation that dengue diseases occur mainly in children although in certain places, like Singapore, this may have changed due to adaptation to vector control measures (Ooi et al., 2001).

Following the bite of an infective female mosquito, the virus undergoes an intrinsic incubation period of 3 to 14 days (average 4 to 7 days) after which the person may experience acute onset of fever accompanied by other non-specific signs and symptoms. During this viraemic period (which may be between 2 to 7 days) the virus circulates in the blood of infected humans. If an uninfected Aedes mosquito feeds on the host during this viraemic period, this mosquito will become infected after an obligatory extrinsic incubation period of 10 to 12 days, it would subsequently be able to transmit the virus to other uninfected hosts. In this transmission cycle, humans are the main amplifying hosts for the virus although studies show that monkeys can get infected and perhaps serve as a source for the virus (Putnam et al., 1995; Gubler et al., 1976; WHO, Fact sheets, 2002).

Dengue virus infection causes a spectrum of illness in humans depending on the infecting virus, the host age and immunological conditions. It may result in asymptomatic illness or ranges from an undifferentiated flu-like illness (Viral syndrome) to dengue fever (DF), to dengue hemorrhagic fever (DHF), and the severe and fatal dengue shock syndrome (DSS) (Nimmannitya, 1993: WHO, 1997).

The World Health Organization has set up standards for the grading of the severity of DHF. There are four grades of severity of which grade III and grade IV are considered to be dengue shock syndrome (DSS).
Grade I: Fever with non-specific constitutional symptoms, and the only haemorrhagic manifestation is a positive tourniquet test and or easy bruising.
Grade II: In addition to the manifestation in grade I, spontaneous bleeding in the forms of skin or other haemorrhages.
Grade III: Circulatory failure manifested by a rapid weak pulse, narrowing of pulse pressure or hypotension with the presence of cold, clammy skin and restlessness.
Grade IV: Profound shock with undetectable blood pressure or pulse (WHO, 1997).

Classical dengue fever is more common in older children, adolescents and adults, and they are less likely to be asymptomatic (Sharp et al., 1995). The fever is abrupt in its onset with high fever, headache, incapacitating myalgias and arthralgias, nausea vomiting and macular or maculopapular rash (Waterman, 1989). The fever usually lasts for 5-7 days and sometimes can follow a biphasic course (Saddle back appearance) (Nimmannitya, 1993).

DHF is primarily a disease of the younger children below 15 years although it may also occur in adults and is mainly associated with secondary dengue infections (Sumarmo et al., 1983; WHO). The critical stage of DHF is at the time of defervescence, when the temperature becomes normal. The major factors that determine the severity of the illness at the time are plasma leakage due to the increased vascular permeability and abnormal homeostasis and other common hemorrhagic manifestations like petechiae, purpuric lesions, and ecchymoses. These symptoms plus a positive tourniquet test are helpful for accurate diagnosis of DHF (Gubler D J., 1998).

DSS is the terminal stage of DHF and is manifested by hypovolaemic shock due to plasma leakage (WHO, 1997). There are four warning signs of DSS: sustained abdominal pain, persistent vomiting, restlessness or lethargy, and a sudden change from fever to hypothermia with sweating and prostration. Early recognition and appropriate treatment by experienced hospital staff can decrease the case fatality rate of DSS to 0.2% but once shock sets in the mortality rate can be over 40% (Nimmannitya, 1994; Rigau-Perez J G, et al., 1998).

The E protein, the largest and the only structural protein exposed on the surface of the virus, is the major protein involved in immunological reactions such as receptor binding, haemmagglutination and neutralization. Infection in humans by one of the serotypes provides life long immunity to that serotype but only temporary protection against other serotypes.

The nucleoplasmid is in turn surrounded by lipid containing the envelope and membrane proteins. In addition to envelope and capsid proteins, dengue virus has seven non-structural proteins NS1, NS2a, NS2b, NS3, NS4a, NS4b and NS5.

### Japanese Encephalitis Virus

Japanese encephalitis is a disease that is spread to humans by infected mosquitoes. It is one of a group of mosquito-borne virus diseases that can affect the central nervous system and cause severe complications and even death.

Japanese encephalitis is caused by the Japanese encephalitis virus, an arbovirus. Arboviruses are a large group of viruses that are spread by certain invertebrate animals (arthropods), most commonly blood-sucking insects. Like most arboviruses, Japanese encephalitis is spread by infected mosquitoes (culex spp).

Those infected develop mild symptoms or no symptoms at all. In those who develop a more severe disease, Japanese encephalitis usually starts as a flu-like illness, with fever, chills, tiredness, headache, nausea, and vomiting. Confusion and agitation can also occur in the early stage. The illness can progress to a serious infection of the brain (encephalitis) and can be fatal in 30% of cases. Among the survivors, another 30% patients showed permanent neurological sequlae.

There are other flaviviruses, which cause viral encephalitis and are within the scope of the present invention. They have a similar disease picture, and they include:
St Louis and Murray Valley Encephalitis
Russian Spring Summer Encephalitis, transmitted by ticks.

Three other encephalitis viruses belong to the Alphavirus genus and are also transmitted by mosquito bite. They are Western, Eastern and Venezuelan equine encephalitis, or WEE, EEE and VEE respectively. These all occur in North and South America. There are also vaccines that protect against EEE and WEE.

Like Japanese encephalitis these viruses often only produce mild general symptoms, similar to mild influenza.

The diagnosis of this disease has previously been made by detecting antibodies in serum and CSF (cerebrospinal fluid) by IgM capture ELISA but this has shown less specificity and the process can take a long time. A vaccine is available but anti-virals are usually ineffective unless administered within hours of being infected, hence treatment is mainly supportive. Japanese Encephalitis virus is not transmitted between humans. Infection with JEV confers life-long immunity.

### Equivalents

The term “equivalent” as used herein and applied to the flavivirus is intended to include similar molecules that can elicit the same or similar response that the flavivirus or a structural or non-structural protein of the flavivirus could elicit. For instance, various antigens expressed by the flavivirus at various stages of infection or various virus particles or fragments may cause similar effects that the whole virus causes. The response may be an immunological response (non-clinical response) or it may be an infectious response (clinical response) or due to vaccination.

### Exposure

The subject may have been exposed to flavivirus but need not show visual symptoms of the infection. The present method detects exposure that may lead to infection (clinical or sub-clinical or non-clinical) or may indicate prior exposure with no symptoms manifested.

The present invention is applicable to detecting exposure to the flavivirus or an equivalent thereof. Exposure may be present or prior exposure to the flavivirus or an equivalent thereof. Preferably, the exposure is sufficient to elicit an immune reaction or response in the body so as to induce a binding partner in response to the flavivirus or equivalent thereof. Once the subject is exposed, the method of the present invention may be applied at any stage of exposure as described above. Preferably, the method is used to detect exposure where there are no signs and symptoms that are obvious of a flavivirus infection. Preferably, the method detects exposure of the subject at any phase of flavivirus infection at an early acute phase for secondary infection or late convalescence stage of exposure to flavivirus or equivalent thereof for the primary infection or vaccination. The exposure may not always manifest in a flavivirus infection or notable signs or symptoms but it will cause a response so as to induce a binding partner. Preferably, the response is an immunological response.

### Immune Response or Immunological Response

An “immune response” or “immunological response” is understood to be a selective response mounted by the immune system of vertebrates in which specific antibodies or fragments of antibodies and/or cytotoxic cells are produced against invading pathogens and antigens which are recognized as foreign in the body.

### Binding Partner

Accordingly, the “binding partner” as used herein is any molecule or cell that is produced against the foreign flavivirus or equivalent thereof. Preferably, it is an immunointereactive molecule. More preferably, the binding partner is an antibody or immunologically active fragment thereof, or a cytotoxic cell. The binding partner includes an immunointeractive molecule that can interact with a flavivirus antigen or equivalent and compete with flavivirus specific immunological agents such as flavivirus specific monoclonal antibodies.

The preferred binding partner is an immunointeractive molecule, which preferably refers to any molecule comprising an antigen binding portion or a derivative thereof. Preferably, the immunointeractive molecule is an antibody against any portion of flavivirus proteins produced during a humoral response in the subject of a flavivirus infection or exposure.

More preferably, the binding partner is an antibody produced in the subject to a flavivirus or related virus components. However, a binding partner of the targeted antibody may also be used. An example of such a binding partner is an anti-idiotypic antibody or an antibody specific for and discriminatory of a subject antibody specific for the member of the flaviviruses or related virus components.

In the early convalescent stages of flavivirus infection antibody preferably IgG derived from previous flavivirus infection is one of the indications of either secondary or primary flavivirus infection. The antibody may be detected by the formation of a complex between it and a component of the member of the flaviviruses. The formation of this immuno-complex with the member of the flavivirus specific IgG and antigen at the flavivirus specific or member specific epitope is indicated by the absence of the attachment of competing flavivirus or member specific immunological agents.

As used herein, an “anti-idiotypic antibody” is an antibody which binds to the specific antigen binding site of another antibody generated in response to exposure to a component derived from the member of the flavivirus genus or immunological relative thereof.

As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′)₂ fragments, and Fv fragments.

Preferably the binding partner is an antibody. More preferably it is a flavivirus IgG molecule or an immunointereactive portion. Most preferably the flavivirus is dengue virus or JE virus.

Cytotoxic cells contained within the biological sample may also serve as binding partners. The cell may interact directly with the flavivirus or any component of a cell lysate from cells infected with flavivirus or an equivalent thereof.

### Biological Sample

The method of the present invention detects exposure to the flavivirus or equivalent thereof via the use of a biological sample obtained from a subject having been potentially exposed to the flavivirus. The biological sample may be any sample from the body that may contain a binding partner. Such biological samples may be selected from the group including blood, saliva, cord fluid, B cells, T-cells, plasma, serum, urine and amniotic fluid. Preferably, the biological sample is serum or plasma or saliva. Most preferably, the biological sample is serum or saliva.

It is also preferred that the biological sample is obtained from subjects suspected of exposure to a flavivirus. A biological sample may also be modified prior to use, such as by dilution, purification of various fractions, centrifugation and the like. Accordingly, a biological sample may refer to a homogenate, lysate or extract prepared from a whole organism or a subset of its tissues, cells or component parts, or a fraction or portion thereof.

It should be noted that a biological sample may also be devoid of a binding partner that can interact with flavivirus or an equivalent thereof. This occurs when the subject has not been exposed to flavivirus or an equivalent thereof. Hence “determining the presence of a complex that forms between a flavivirus specific binding partner present in the biological sample and an anti-flavivirus IgA captured component” may yield a zero result as a complex cannot form in the absence of binding partners. The only complex that may form in this instance would comprise the competing flavivirus specific immunological agent such as a monoclonal antibody designed to compete with binding partners in the biological sample.

Reference to a biological sample being placed in contact with a component of a lysate, preferably an immunogenic component of flavivirus or its immunological relative thereof should be understood as a reference to any method to facilitating the interaction of one or more immunointeractive molecules of the biological sample with a component of the flavivirus or its immunological relative thereof. The interaction should be such that coupling or binding or otherwise association between the immunointeractive molecule and a specific immunogenic component of the flavivirus or its immunological relative thereof can occur.

The biological sample is contacted with a mixture of anti-flavivirus IgA captured viral components of flavivirus or equivalent thereof.

### Anti-Flavivirus IgA Captured Component.

Anti-flavivirus IgA is used to capture flavivirus or member specific components. Preferably the component is an immunological component that is reactive to a flavivirus specific binding partner and a competing flavivirus specific immunological agent. The anti-flavivirus IgA may be obtained by any methods available to the skilled addressee for making antibodies to an antigen. Preferably, the IgA is captured by anti-human IgA.

The present invention specifically uses anti-flavivirus IgA captured components as opposed to IgG or IgM which generally results in loss of sensitivity of the test. It has now been found that the use of IgA to capture flavivirus components can improve the sensitivity of the serological test. IgA can capture flavivirus components react more specifically with binding partner from recent infection and exposure.

When a subject is exposed to flavivirus, the body immune system reacts to initially remove the virus. This causes a chain of events generally manifesting in an immunological response to the plethora of antigens presented by the flavivirus or an equivalent thereof. Hence the component provided in the present invention represents any stage of exposure to the flavivirus and may be an antigen or portion of the antigen that includes an epitope either specific to all flavivirus or to the serotype. The components may be derived from any source including the flavivirus itself. However, it is preferred that the component is derived from a cell infected with the flavivirus virus. This may be derived from a cell culture or from tissue or biological samples infected with the flavivirus. Most preferably, the components are derived from a culture of cells infected with the flavivirus from which a cell lysate is obtained.

In the present invention the cell lysate preferably comprises a mixture of flavivirus immunogens, which includes virus particles and both the structural and non-structural viral proteins. These immunogenic components are captured by anti-flavivirus IgA. Preferably the flavivirus immunogens are immunogenic components of the lysate that are capable of eliciting an immunological reaction to a flavivirus specific binding partner present in the biological sample and to competing flavivirus specific immunological agents. The lysate provides viral components, preferably immunogenic components that may be provided by the flavivirus at any stage of its development.

The lysate of the present invention may be obtained from any source of cells that have been infected with the specific member of the flaviviruses or equivalent thereof. Preferably, the cells are infected in an in vivo culture with flavivirus or equivalent thereof.

Any type of cell may be infected. Preferably, the cell type is capable of infection and culture of flavivirus. However, it is preferred that cells capable of producing high titres of flavivirus are infected in accordance with the methods of the present invention, including, but not limited to, continuous cell lines commonly available (e.g. Vero cells (Vero-PM strain), CV-1 cells, LLC-MK2, C6/36 and AP-61 cells), primary cell lines such as fetal Rhesus lung (FRhL-2) cells, BSC-1 cells, and MRC-5 cells, or human diploid fibroblasts. A combination of cell types is also envisaged by the present invention. C6/36 or AP-61 cells are infected with flavivirus or equivalent. Most preferably the cell type is C6/36.

Cells may be cultured for any period, preferably for a period which allows the flavivirus to establish and infect the cell. More preferably, the cells are cultured until a cytopathic effect is apparent in the cell culture thereby indicating active infection of the virus in the cells.

At this point, the cells may be lysed by any method available to the skilled addressee. Preferably, the use of a hypotonic buffer including a detergent such as Triton X 100 may be used providing the lysing buffer does not affect the immunogens of the flavivirus or equivalents thereof but preferably inactivates the live virus particles.

It should be appreciated that the lysate will contain a mixture of viral immunogenic components including structural and non-structural flavivirus virus antigens as well as whole flavivirus virus particles. For the dengue virus these may be selected from the group including DEN1, 2, 3 or 4. The present invention seeks to identify a mixture of antigens by a mixture of binding partners that are generated in the biological sample in response to exposure to the flavivirus or an equivalent.

Preferably the flavivirus is dengue virus or JE virus.

More preferably the flavivirus or dengue specific immunogenic component is a structural or non-structural protein of the flavivirus or dengue virus. More preferably, the structural protein is selected from the group including C-Capsid, M-Membrane and E-Envelope proteins, which may be captured by anti-flavivirus IgA. More preferably, the non-structural proteins for the dengue are selected from the group including NS1, NS2a, NS2b, NS3, NS4a, NS4b and NS5.

The lysate may be processed in any manner. Preferably, the lysate is clarified to remove nuclei and cellular debris and whole flavivirus particles. The lysate may be aliquoted and stored at −70° C. to −80° C. for future use.

For the dengue virus previous methods used specific dengue antigens DEN 1, 2, 3 and 4 (present in the supernatant of dengue virus infected cells) to detect antibodies indicative of dengue virus infection. However, the present invention does not solely use these antigens but a mixture of flavivirus molecules/immunogens (present in the flavivirus infected cells, which may contain flavivirus particles and other immunological components, preferably structural and non-structural proteins) against which antibodies develop in the course of a flavivirus exposure.

### Competing Flavivirus Specific Immunological Agent

The immunological agent, preferably an antibody or monoclonal antibody is used to compete with the binding partner present in the biological sample. The immunological agent is also reactive to flavivirus specific components. Any immunological agent that is specific for flavivirus or more preferably to an epitope of the flavivirus is preferred. Generally the immunological agent will be specific to the epitope on the envelope portion of the flavivirus virus. Most preferably, the competing flavivirus specific immunological agent is specific for an anti-flavivirus IgA captured component.

Since the immunological agent is to compete with the flavivirus specific binding agent, it is also preferred that the immunological agent and the flavivirus specific binding agent compete for at least one or the same epitope. Accordingly it is preferred that the immunological agent is specific to an epitope of the anti-flavivirus IgA captured components. A combination of the IgA captured components and a highly specific immunological agent will enhance the specificity of the test to the member of the flavivirus genus.

Specificity in the test may be enhanced by the degree of specificity of the competing flavivirus specific immunological agents. Preferably the competing flavivirus specific immunological agent is an antibody such as a polyclonal antibody or a monoclonal antibody. More preferably it is a monoclonal antibody specific for an epitiope of the flavivirus, more specifically to an anti-flavivirus IgA captured component.

By knowing the antigen to which the antibody is raised, the antigen of the component can be identified.

Any methods available to the skilled addressee may be used to make an antibody or monoclonal antibody to the flavivirus virus.

### Complex Formation

The components and the biological sample are contacted so that a complex may form between the components and the binding partner present within the biological sample. Preferably, immunogens of the flavivirus particles, including but not limited to those of the structural and non-structural proteins captured by anti-flavivirus IgA having an epitope specific to flavivirus, will form complexes with either binding partners or a competing flavivirus specific immunological agent. Preferably, the specific binding partners are antibodies or fragments thereof present in the biological sample. These will only be present when the subject has been exposed/immunized to the flavivirus.

Preferably, the complex will form between an antibody, preferably an IgG specific for the member of the flavivirus genus or equivalent thereof and a anti-flavivirus IgA captured flavivirus viral component.

The binding partner may also be a cytotoxic cell such as a cytotoxic T-cell from the biological sample that reacts to the flavivirus immunogens.

A competing flavivirus or member specific immunological agent will also form a complex with the component if the same epitope is remained free on the component. Where the binding partner and the immunological agent are specific for the same epitope, a competition will arise manifesting in an indication of the presence of the binding partner and prior exposure to flavivirus.

The preferred method of the present invention relies upon the detection of competing flavivirus or member specific immunological agent with flavivirus specific binding partners present in the biological sample. The competing immunological agent and flavivirus or member specific binding partner compete for a component of the flavivirus antigen present in cell lysate derived from a cell infected with the flavivirus or an equivalent thereof which has been captured using anti-flavivirus IgA. The complex may comprise one or more binding partners bound to one or more components derived from flavivirus or an equivalent thereof.

The biological sample is left in contact with the component derived from flavivirus or an equivalent thereof for a period of time sufficient and conditions, which allow the stable formation of a complex and inhibits the attachment of competitive immunological agent such as a monoclonal antibody specific for flavivirus or any particular member of the genus.

Once attached, the competing flavivirus specific immunological agent may be added. Therefore, it is preferred to have a pre-incubation step where the binding agent and component are allowed to form a complex prior to the addition of the immunological agent. However, these components may also be added simultaneously.

The invention preferably utilizes a flavivirus specific or any particular member specific antibody to compete with binding partners such as anti-flavivirus specific or any particular member specific IgG from the host body for an epitope either specific to flavivirus or any particular member of the genus or to their serotypes present on the envelope protein of flavivirus derived from a cell lysate, which includes a mixture of flavivirus immunogenic components including flavivirus particles amongst other antigens indicative of flavivirus infection. The methods and kits of the present invention seek to detect components and binding partners, which form complexes and are indicative of a flavivirus infection. These components and binding partners are generated in the course of a flavivirus infection.

In yet another aspect, the biological sample may be applied to a solid support such as, but not limited to, a nitrocellulose membrane or polystyrene plate coated with anti-flavivirus IgA. The solid support may also have the component captured by anti-human IgA and derived from flavivirus or an equivalent thereof applied to it. The component derived from a flavivirus or equivalent thereof is then left in contact with the biological sample for a period of time sufficient and under conditions, which allow the stable formation of a complex in the presence of competing immunological agents specific to flavivirus or any members of the genus. Once a complex is formed, a detection system is then added to facilitate the detection of the specific binding of the immunological agent in the complex.

### Determining the Presence of a Complex

Detecting the complex between the anti-flavivirus IgA captured flavivirus viral components derived from flavivirus and a flavivirus specific binding partner such as an immunointereactive molecule or flavivirus specific immunological agent, may be based on any convenient method, which will be known to those of the skill of the art.

It is contemplated that procedures useful for detecting anti-flavivirus IgA captured components, binding partners and flavivirus specific immunological agents such as monoclonal antibodies which form complexes and are indicative of a flavivirus infection in a biological sample include, but are not limited to, immunological assays, such as immunoblotting, immunocytochemistry, immunohistochemistry or antibody-affinity chromatography, Western blot analysis, or variations or combinations of these or other techniques such as are known in the art.

In a preferred embodiment, the method of detection employs a further detection agent such as specific antibodies and anti-antibodies conjugated with enzyme, or simply using an immunogenic agent directly bound to a reporter group such as an enzyme. A suitable enzyme is horse radish peroxide (HRP) which permits detection of the complexes formed with competing components. For added specificity, monoclonal antibodies may be employed.

The present invention includes the use of a flavivirus or flavivirus member specific immunological agent that has a reporter group. The use of this enhances the speed of which the test can be performed. It reduces the number of steps required to identify complex formation. A suitable reporter group is an enzyme such as HRP. Other suitable reporter groups may be used and are familiar to those skilled in the art.

The complex formed by a component of the cell lysate and the binding partner may be detected using a detection agent that contains a reporter group and specifically binds to the component/binding partner complex. Such detection agent may comprise, for example, flavivirus specific or member specific antibody derived from other species of animals or other agents that specifically bind to the binding partner, such as an anti-immunoglobulin (i.e. antibody), protein G, protein A or a lectin. Alternatively, a competitive assay may be utilized, in which a detection agent capable of binding an antigen derived from the member of flaviviruses, is labeled with a reporter group and allowed to bind to the immobilized component of the cell lysate in combination with the binding partner of the biological sample. The extent to which the binding partner of the biological sample inhibits the binding of the labeled flavivirus or flavivirus member specific detection agent to the immobilized component is indicative of the reactivity of the binding partner of the biological sample with the immobilized component.

In a preferred embodiment, the detection reagent is an antibody or secondary antibody or an antigen-binding fragment thereof, capable of binding to the binding partner of the biological sample. Antibodies may be prepared by any of a variety of techniques known to those of ordinary skill in the art (See, for example, Harlow and Lane, Antibodies: A Laboratory Manual, Cold Spring Harbor Laboratory, 1988). In general, antibodies can be produced by cell culture techniques, including the generation of monoclonal antibodies, or via transfection of antibody genes into suitable bacterial or mammalian cell hosts, in order to allow for the production of recombinant antibodies.

The secondary antibody, which may be conjugated to a label, can be added to the complex to facilitate detection. A range of labels providing a detectable signal may be employed. The label may be selected from a group including chromogen, an enzyme, a catalyst, a fluorophore and a direct visual label. In the case of a direct visual label, use may be made of a colloidal metallic or non-metallic particle, a dye particle, an enzyme or a substrate, an organic polymer, or a latex particle. A large number of enzymes suitable for use as labels are disclosed in U.S. Pat. Nos. 4,366,241, 4,843,000 and 4,849,338. Suitable enzyme labels in the present invention include alkaline phosphates, horseradish peroxidase, preferably horseradish peroxidase. The enzyme label may be used alone or in combination with a second enzyme which is in solution. In the present invention a secondary antibody attached with horseradish peroxidase, which then reacts with its substrate OPD or DAB and produces a visually detectable color change, preferably achieves the detection of the complex that formed with immunogens and competitive flavivirus or flavivirus member specific immunological agents such as monoclonal antibodies specific to flavivirus or any member of the genus.

### Presentation of the Anti-Flavivirus IgA Captured Components

In a preferred embodiment, the methods as herein described involve the use of a cell lysate derived from a cell infected with a flavivirus or a flavivirus member of the genus, or the anti-flavivirus IgA captured components (herein referred to interchangeably as the “components” or the “components of the cell lysate”), immobilized on a solid support such as a polystyrene or nitrocellulose membrane to which a binding partner present in a biological sample may bind.

Accordingly in another aspect of the present invention there is provided a solid support for use in a method for detecting exposure of a subject to a flavivirus or equivalent thereof, said method comprising:
contacting a biological sample from the subject with a mixture of anti-flavivirus IgA captured components of flavivirus or an equivalent;
subjecting the biological sample and the anti-flavivirus IgA captured components to a competing flavivirus specific immunological agent
determining the presence of a complex that forms between a flavivirus or flavivirus member specific binding partner present in the biological sample and an anti-flavivirus IgA captured component in the presence of the competing flavivirus specific immunological agent;
   said support comprising anti-flavivirus IgA captured components immobilized on the support.

The solid support may be any material known to those of ordinary skill in the art to which a binding partner or component of the cell lysate may be attached. For example, the solid support may be a test well in a microtitre plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The binding partner or the component of the cell lysate may be immobilized on the solid support using a variety of techniques known to those of skill in the art, which are amply described in the patent and scientific literature. In the context of the present invention, the term “immobilization” refers to both immuno-absorption or non-covalent association, such as adsorption, and covalent attachment (which may be a direct linkage between the antigen or nucleotide and functional groups on the support, or may be a linkage by way of a cross-linking agent). Immobilization by immuno-absorption with antibody coated to a well in a microtitre plate or a simple absorption to a membrane is preferred. In such cases, adsorption may be achieved by contacting the binding partner or a component of the cell lysate, in a suitable buffer, with the solid support previously coated with antibody for a suitable amount of time. The contact time varies with temperature, but is typically about 1 hour and over night.

Immuno-attachment of a binding partner, an anti-flavivirus IgA captured component or a flavivirus or member specific immunological agent to a solid support may also be achieved by first reacting the support with a bifunctional reagent that will react with both the coated antibody and a immunogenic component such as non-specific epitope, of a binding partner or a component. For example, the binding partner, an anti-flavivirus IgA captured component or a flavivirus specific immunological agent may be immunologically attached to supports having an appropriate antibody coating using anti-antibody.

### General Description of the Process

The following is a general description of the method merely to illustrate certain preferred embodiments but the invention should not be restricted to this general description nor to the dengue virus. The dengue virus is merely a preferred flavivirus for the purposes of illustration a flavivirus for the invention.

The assay may be conducted as a two-step sandwich assay. This assay may be performed by first contacting a dengue specific binding partner present in a biological sample that has been immobilized on a solid support (this may be the well of a microtitre plate), with the component of a cell lysate as herein described, such that a component is allowed to bind to the immobilized binding partner. Unbound sample is then removed from the immobilized complex and a detection reagent (preferably a second antibody capable of binding to the binding partner or the component, containing a reporter group) is added. The amount of detection reagent that remains bound to the solid support is then determined using a method appropriate for the specific reporter group.

More preferably, once the binding partner or a component of the cell lysate is immobilized on the support as described above, the remaining binding sites on the support are typically blocked. Any suitable blocking agent known to those of ordinary skill in the art, such as bovine serum albumin or skin milk with either Triton X 100 or Tween 20 ™ (Sigma Chemical Co., St. Louis, Mo.) The immobilized binding partner or component is then incubated with a component of a cell lysate or binding partner, respectively, such that a complex between the binding partner and the component is allowed to form. The component or binding partner may also be diluted with a suitable diluent buffer, such as phosphate-buffered saline (PBS) with dengue antibody negative human serum and either Triton X 100 or Tween 20 prior to incubation. In general, an appropriate contact time (i.e., incubation time) is a period of time preferably 30 minutes that is sufficient to allow a component of the cell lysate to bind to the immobilized binding partner, or vice versa and then competitive immunological agent such as an antibody to dengue virus, more preferably a monoclonal antibody is added to the mixture for the competition assay and is allowed to work for another 30 minutes. The competitive immunological agent may have a reporter group directly bound to it. Preferably, the contact time is sufficient to achieve a level of binding to the target epitope on the attached dengue antigen that is at least about 95% of that achieved at equilibrium between the bound and unbound binding partner or component of the cell lysate. Those of ordinary skill in the art will recognize that the time necessary to achieve equilibrium may be readily determined by assaying the level of binding that occurs over a period of time. At room temperature, an incubation time of about 30-60 minutes is generally sufficient.

Unbound components may then be removed by washing the solid support with an appropriate buffer, such as PBS containing 0.05% Tween 20™ or Tween 80. A detection agent which is capable of binding to the component of the cell lysate or the binding partner, and which contains a reporter group, may then be added to the solid support. The detection agent is then incubated with the immobilized binding partner-component complex for an amount of time sufficient to detect the bound component or binding partner. An appropriate amount of time may generally be determined by assaying the level of binding that occurs over a period of time. Unbound detection agent is then removed and bound detection agent is detected using the reporter group. The method employed for detecting the reporter group depends upon the nature of the reporter group.

Alternatively, a reporter group may be bound directly to the immunological agent. The reporter group can then be detected immediately. For radioactive groups, scintillation counting or autoradiographic methods are generally appropriate. Spectroscopic methods may be used to detect dyes, luminescent groups, chromogenic enzymes and fluorescent groups. Chromogenic enzymes include, but are not limited to, peroxidase and alkaline phosphatase. Fluorescent groups include, but are not limited to, fluorescein isothiocyanate (FITC), tetramethylrhodamine isothiocyanate (TRITC), rhodamine, Texas Red and phycoerythrin. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products. The substrate can be selected from a group of agents consisting of 4-chloro-I-naphtol (4CN), diaminobenzidine (DAB), aminoethyl carbazole (AEC), 2,2′azino-bis(3-ethylbenzothiazoline-6-sulfonic acid) (ABTS), ophenylenediamine (OPD) and tetramethyl benzidine (TMB).

It may also be desirable to couple more than one reporter group to a detection agent. In one embodiment, multiple reporter groups are coupled to one detection agent molecule. In another embodiment, more than one type of reporter group may be coupled to one detection agent. Regardless of the particular embodiment, detection agents with more than one reporter group may be prepared in a variety of ways. For example, more than one reporter group may be coupled directly to a detection agent, or linkers that provide multiple sites for attachment can be used.

In a related embodiment, the method as herein described may be performed in a polystyrene plate or flow-through or strip test format, wherein the binding partner of a biological sample or a component is immobilized by anti-dengue antibody or on a membrane, such as nitrocellulose. In the flow-through test, for example, a component is capable of binding to the immobilized binding partner as the sample passes through the membrane. Alternatively, a binding partner present in a biological sample is capable of binding to the immobilized component of the cell lysate as the sample passes through the membrane. A second, labeled detection agent then binds to the binding partner-component complex as a solution containing the detection agent flows through the membrane. The detection of bound detection agent may then be performed as described above.

In the strip test format, one end of the membrane to which a component of the cell lysate is bound is immersed in a solution containing the biological sample. The binding partner in the biological sample migrates along the membrane through a region containing a detection agent and to the area of the immobilized component. The concentration of detection agent at the area of immobilized binding partner-component complex indicates the presence of binding agent in a biological sample. Typically, the concentration of the detection reagent at that site generates a pattern, such as a line, that can be read visually. The absence of such a pattern indicates a negative result. In general, the amount of binding partner immobilized either on the membrane or polystyrene plate is selected to generate a visually discernible pattern when the biological sample contains a level of a binding agent that would be sufficient to generate a positive signal in the sandwich assay, in the format discussed above. Such tests can typically be performed with a very small amount of biological sample.

In a simpler version of the strip test or dipstick test, components of the cell lysate can be immobilised onto a membrane, such as a nitrocellulose membrane. Strips of membrane may then be subjected to biological samples to form complexes between the components and a binding partner present in the biological sample. The complex is then detectable by any means described above using a detection agent, such as a monoclonal antibody. The dipstick test can provide a quick indication of previous exposure without using large biological samples.

As used herein, “binding” refers to a non-covalent association or immunological bonding between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant may be determined using methods well known in the art.

Solid supports contemplated by the method and kits of the present invention include any surface to which either the binding partner, components derived from the flavivirus or any specific member of the group or equivalent thereof or a competing flavivirus specific immunological agent can immobilize using immuno-absorption. Examples of surfaces include without being limited to, polystyrene or membranes including nitrocellulose membranes, polytetrafluorethylene membrane filters, cellulose acetate membrane filters and cellulose nitrate membrane filters with filter paper carriers. Most preferably, the membrane is polystyrene and the membrane is nitrocellulose membrane.

Alternatively, the diagnostic methods of the present invention may adopt an automated analytic method using a biological microchip. For instance, a diagnostic kit can be structured to perform immunoblotting using a glass slide coated with the component of the cell lysate. This diagnostic kit may comprise a biological microchip onto the surface of which a components of the cell lysate is immobilized, an appropriate buffer, a standardised sample comprising a detectable level of binding agent, and a secondary detection reagent, as herein described.

The method and kits of the present invention can detect specific exposure of human or animals to a flavivirus or any specific member of the family or equivalent thereof either during acute infection or in convalescent phase. As used therein “acute infection” refers to the period of time when a virus has infected a host and is actively replicating and/or causing symptoms associated with infection like fever, rush, joint pain and or abdominal pain. The “convalescent phase” refers to the stage of flavivirus infection cycle when flavivirus virus is no longer multiplying or remains in the host blood and has developed binding partners such as, but not limited to antibodies. Using the method and kit of the present invention, exposure can be detected at any time after generation of a binding partner in the infected patient or patient derived it from his/her previous infection/infections.

### Kits

In another aspect of the present invention there is provided a kit for detecting exposure of a subject to a flavivirus or any member of the family or equivalent thereof, said kit comprising;
anti-flavivirus IgA captured components from flavivirus;
competing flavivirus specific immunological agent; and
at least one detection agent for detecting a complex that forms between a binding partner present in a biological sample and an anti-flavivirus IgA captured component in the presence of the competing flavivirus specific immunological agent.

Optionally, the kit will also include additional parts such as washing buffers, incubation containers, blocking buffers and instructions as are necessary for conducting the method.

Accordingly the present invention provides a kit for detecting exposure of a subject to flavivirus or any member of the family or an equivalent thereof. The kit may be any convenient form which allows for a binding partner in a biological sample to interact with an anti-dengue IgA captured flavivirus viral component and further compete with a competing flavivirus specific immunological agent. The result is an indication, by the presence of flavivirus specific or flavivirus specific-binding partners in the biological sample, of prior exposure to flavivirus. Preferably the kit comprises a solid support such as described herein adapted to receive or comprise anti-flavivirus IgA captured components of flavivirus or an equivalent thereof. The kit may also comprise reagents, reporter molecules capable of providing detectable signals and optionally instructions for use. The kit may be in modular form wherein individual components may be separately purchased.

The kit may be a modular kit comprising one or more members wherein at least one member is a solid support comprising a anti-flavivirus IgA captured flavivirus component of flavivirus or equivalent or cell lysate comprising an immunogenic component derived from a flavivirus or equivalent thereof.

In an alternative embodiment the solid support comprises an array of binding partners for one or more components of one or more flavivirus or equivalent thereof from one or more subjects.

The present invention also provides individual components of the kit for use in the method of the present invention. The invention provides solid supports which include anti-flavivirus IgA captured components of the flavivirus for use in the detection of exposure to the flavivirus. In one embodiment, the invention provides a polystyrene 96 well plate or a nitrocellulose membrane to attach viral antigen, either for use as an immobilized anti-flavivirus IgA captured flavivirus viral components or as a dot blot or use as a dip stick, which includes components of the flavivirus or equivalent thereof. Preferably, the plate or membranes include components selected from the group including flavivirus structural and non-structural proteins, flavivirus particles and fragments thereof, glycoproteins, lipids and carbohydrates derived from the flavivirus or any mixture thereof.

The solid support may also be a microtitre plate, glass slide or biological microchip wherein the components of the cell lysate are immobilised. These solid supports can then be subjected to the biological sample to detect flavivirus exposure. Preferably polystyrene microtitre plate is used to attach flavivirus antigen by immuno-purification using anti-flavivirus IgA from flavivirus infected cell lysate.

In the case of a nitrocellulose membrane, the second support may be a holder, which holds the solid support to improve manipulation of the solid support, which has immobilised components of the flavivirus. For instance, the nitrocellulose membrane may be supported on a stick that enables the membrane to be dipped into a biological sample such as serum. This is useful as a component of a kit since small amounts of biological sample can be tested simultaneously.

### Assessing Relative Risk of Infection

In yet another aspect, the present invention also provides a method of assessing the relative risk of one or more subjects being exposed to flavivirus or any member of the family or an equivalent thereof within a defined location (e.g. geographical area, housing estate, means of transport or center for medical treatment or assessment), comprising;
a) obtaining samples from a representative population within a defined location; and
b) assessing exposure of individual members of a sample population to a flavivirus or any member of the family or equivalent thereof by the method comprising the steps of—
   i) contacting a biological sample from a subject with an anti-flavivirus IgA captured component derived from the flavivirus or equivalent thereof to form a complex between the component and a flavivirus specific binding partner present in the biological sample;
   ii) determining the presence of the complex that forms between a flavivirus specific binding partner present in the biological sample and an anti-flavivirus IgA captured component in the presence of a competing flavivirus specific immunological agent, wherein the presence of the complex is indicative of exposure of the subject to a flavivirus or any member of the family or equivalent thereof; and
   iii) assessing the relative risk of exposure within the defined location.

Risk analysis may be conducted using software in a computer readable form. Consequently, the present invention further relates to a computer readable program and computer comprising suitable for analysing exposure of subjects or group of subjects or a risk of exposure of subject or group of subjects to a flavivirus or equivalent thereof.

The method or technique of the present invention allows for the epidemiological study or sero-surveillance of outbreaks of infection caused by flavivirus or any member of the family or equivalent thereof. Such studies provide valuable information, which advance multiple facets of research in the area of flavivirus disease. For example, epidemiological studies aid in the identification of the index of an infection. Such information enables the identification of a defined location from which the source of virus responsible for a viral outbreak originated.

Additionally the technique/method of the present invention permits for the rapid identification or isolation of subjects who are infected with a flavivirus or equivalent thereof without major laboratory equipment or even in field conditions. Such information aids in identifying subjects, who require medical treatment as well as defining locations that require further investigation or disease control approaches such as identification of breeding places and its control. Further, the technique of the present invention allows for the monitoring of an infected patient to determine the presence of—anti-flavivirus specific IgG. Alleviation of IgG titre or its presence in an early phase of infection may be the indication of secondary infection and thus, help the monitoring of the subsequent phases of flavivirus infection like dengue hemorrhagic fever (DHF) or dengue shock syndrome (DSS).

Further, the technique of the present invention provides a means for identifying subjects who are infected with any specific member of the genus of flavivirus and serotypes involved, allowing for the rapid detection, risk of further infection, pointing to the location of an infection and disease control strategy.

A reference herein to a patent document or other matter which is given as prior art is not to be taken as an admission that the document or matter was known or that the information it contains was part of the common general knowledge as at the priority date of any of the claims.

Examples of the procedures used in the present invention will now be more fully described. It should be understood, however, that the following description is illustrative only and should not be taken in any way as a restriction on the generality of the invention described above.

### EXAMPLES

### Example 1

### C-Elisa for the Detection of Dengue Specific IgG

### 1. Materials and Methods

1.1 Preparation of viral lysate antigens for C-ELISA: Lysate dengue viral antigens were prepared against all four serotypes of dengue virus according to the method described by Cardosa et al., 2002. Briefly, dengue viruses (5 m.o.i.) were grown in C6/36 cells with virus maintenance medium containing 2% fetal calf serum were incubated 4-5 days depending on the development of cytopathic effects and serotypes of the virus. The medium was decanted and the flask with infected cells was washed four times with PBS, treated with 1 ml hypotonic buffer with 1% trix100 for an hour and finally centrifuged at 14000 rpm for 10 minutes. The supernatant was collected tested against dengue group specific and serotype specific monoclonal antibodies by indirect ELISA, and allocated 500 μl in eppendrofs and stored at −70° C. until use.

1.2 Monoclonal antibodies: Pan dengue specific MAbs were obtained from two different sources (Abcam, Oxford, UK and Immunology Consultants Limited, USA) and both MAbs were certified by CDC, Atlanta, USA. Dengue serotypes specific MAbs (den-1, den-2, den-3 and den-4) were obtained from ICL, USA. The reactivities of all MAbs against four serotypes of Singapore isolates were also evaluated by indirect ELISA and dot-blot immunoassay.

1.3 Test sera: A total 360 serum samples were used in this study. These samples were received from different clinics and hospitals for the diagnosis of dengue infection using either PCR or virus isolation or serological assays. The presence of dengue reactive antibodies (IgG) in the sample was initially screened using two types of dengue virus IgG indirect ELISAs (Pan-Bio, Australia and IVD Research Inc, Carlsbad, Calif. 92008, USA) and level of IgG was then determined using dot-blot EIA by serial dilutions. All samples were preserved at −80° C. until they were tested by C-ELISA.

1.4 Indirect ELISA: Three types of indirect ELISAs were used in this study. The commercial kits (Pan-Bio, Australia and IVD Research Inc, Carlsbad, Calif. 92008, USA) were used for the screening of dengue virus reactive IgG antibodies in serum samples and tests were performed according to the procedures described by the manufactures. In house indirect ELISA was used to evaluate the MAbs and to determine the optimal viral antigen, MAb and serum dilutions for use in the competitive assay. Indirect ELISA was performed in 96 well maxi-sorb polystyrene plates. The maxi-sorb 96-well flat bottom microtiter plates (NUNC, Denmark) and volumes of 100 μl of reagents were used throughout the assay. ELISA plates were coated with 100 μl per well of anti-human IgA at 1:500 dilution in carbonate/bicarbonate buffer (Fluka, Biochemika, Switzerland) buffer at pH 9.6 and incubated either at 37° C. for 1 hour or overnight at 4° C. The plates were washed one time with washing buffer (PBS containing 0.05% Tween 20) and 100 μl of anti-dengue IgA at a predetermined optimal dilution (1:5000) in PBS was added to each well. The plates were incubated for 1 hour at 37° C. and washed again one time and 100 μl of dengue lysate antigens (Den-1 to Den-4) at predetermined dilution of 1:100 in PBS was added to each well and again incubated at 37° C. for a 1 hour. Following incubation, plates were washed four times and MAbs were added at serial dilutions from 1:250 to 64,000 in C-ELISA diluent buffer (PBS with 0.3% dengue antibodies negative human serum and 0.1% Triton x100) and incubated at room temperature for 30 minutes. After six times washing, 100 μl of goat anti-mouse IgG conjugated with horseradish peroxidase (HRP) added to each well at 1:3000 dilution in diluent buffer and incubated at room temperature for another 30 minutes. The optimum dilutions of goat anti-mouse IgG with HRP 1:3000, had been determined by direct ELISA after plates were coated with serial dilutions of MAb. Following incubation, plates were washed six times and 100 μl of OPD (othro-phenelyne-diaimine, Sigma, USA) was added to each well and incubated at room temperature for 5-10 minutes. Further colour development was stopped after 5-10 minutes by adding 100 μl 2 M H₂SO₄ and optimal density was read at a 492-nm wavelength.

1.5 Competitive ELISA (C-ELISA): C-ELISA depends on the blocking of the binding of MAb to a dengue virus common epitope in the presence of dengue positive serum. Competition is detected as a reduction in the optimal density reading obtained with the MAb alone. The amount of decrease in ELISA signal would be proportional to the titer of dengue virus specific IgG in the serum. The test was carried out by three ways, namely, simultaneous addition of test serum and MAb to antigen capture wells leading to a competitive ELISA or addition of MAb after pre-incubation of half an hour with test serum or one hour pre-incubation with serum washing six times followed by addition of MAb which called blocking ELISA or B-ELISA. All three formats were tested in this study.

Four dengue serotypes isolated in Singapore were used in this study. MAbs used in this study were screened using more than 100 isolates (Den-1, Den-2, Den-3 and Den-4), isolated over last four years (2002-2005) and found reacts equally with all. The optimal dilution of lysate antigens were determined by checkerboard titration using antigen capture technique on wells previously captured with anti-dengue IgA and 1:100 dilution of lysate antigen was optimal for the assay.

Maxi-sorb 96-well flat-bottom microtiter plates were used to capture antigen using the method described above, washed four times and plates were ready to use for C and B-ELISA assays. For the assay 45 μl of blocking buffer (PBS with 0.3% dengue antibodies negative human serum and 0.1% Triton x100) was added to each well and then 5 μl of test and control sera were added to the respective wells and are used according to the assay: for simultaneous C-ELISA 50 μl of specific MAb diluted 1:1000 in blocking buffer was added to wells containing serum; for pre-incubation C-ELISA, specific MAb was added after 30 minutes of incubation serum at room temperature and for B-ELISA serum was incubated for an hour at room temperature, washed six times and specific MAB was added to each well. After incubations (60 minutes for simultaneous and 30 minutes for pre-incubation C-ELISAs, plates were washed 6 times, and 100 μl of a 1:3000 dilution of HRP-conjugated goat anti-mouse IgG diluted in blocking buffer was added to each well. The rest of the assay was done exactly as described for indirect ELISA. In case of B-ELISA, following addition of test sera plates were incubated at RT for an hour washed 6 times and then added 100 μl of MAb to each well at 1:1000 dilutions. The rest steps were similar to C-ELISA.

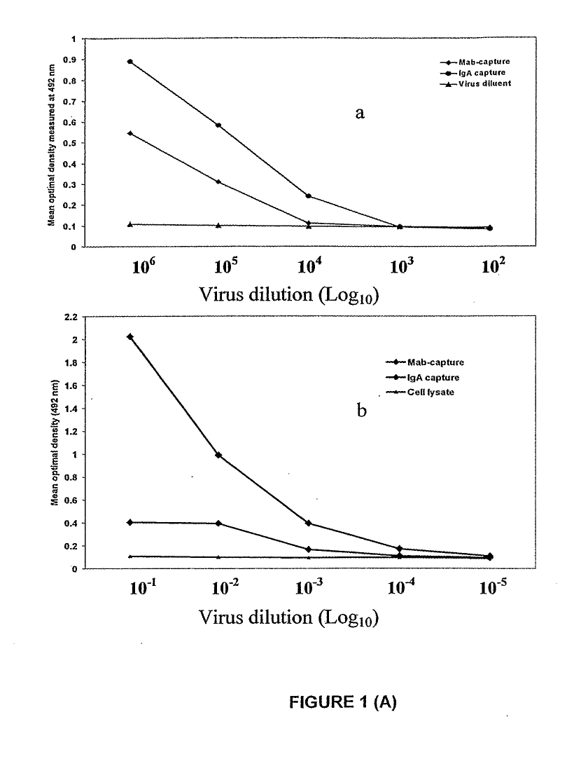

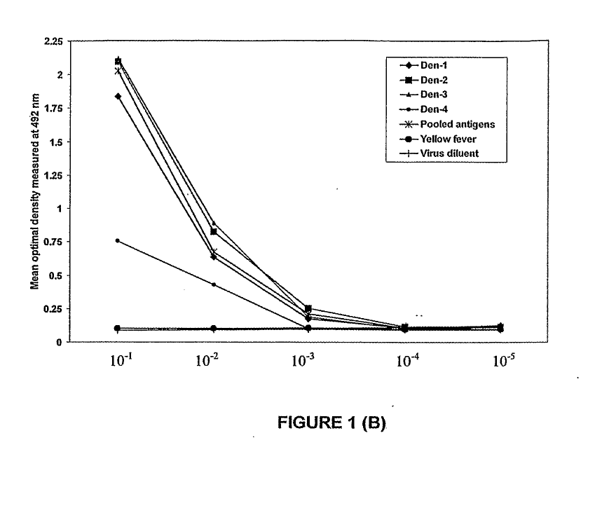

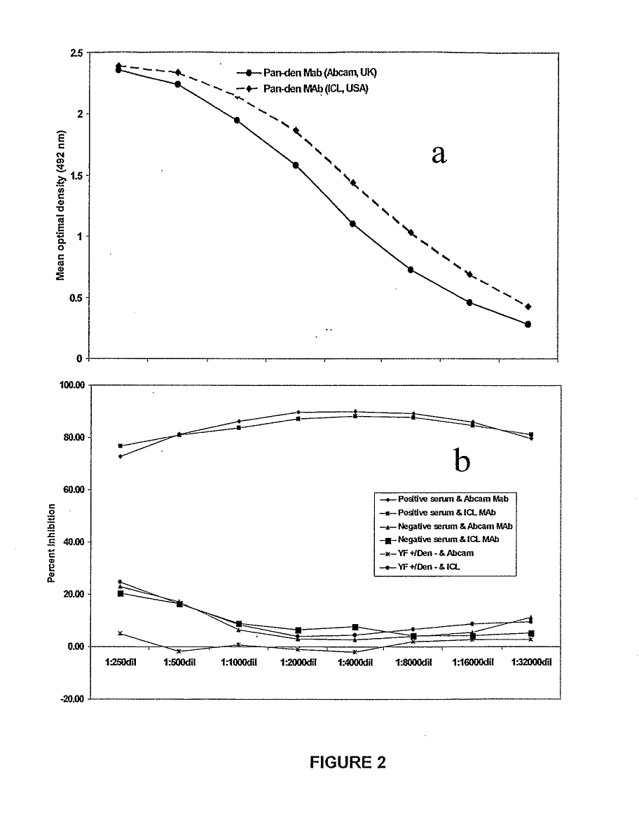

1.6 Establishment of optimal MAbs and serum dilutions and test format: For initial establishment of test parameters, two dengue virus-2 neutralizing positive sera (1:640 and 1:160), two convalescent dengue sera (confirmed by PCR), two yellow fever neutralizing positive sera (1:1280 and 1:80) and two flavivirus negative sera (VNT=1:10) were used. First, the MAbs were titrated by adding serial dilutions (1:250 to 1:64000) in blocking buffer to antigen-captured plates and running an indirect ELISA (FIG. 1a and 2b). Then, two dilutions of MAbs were arbitrarily chosen, corresponding to 100 and 75 to 80% of the plateau OD on the titration curve and tested by C-ELISA and B-ELISA against serial fold dilutions of sera (FIG. 2a, 2b and 3). All samples were tested in duplicate. Controls included wells with no serum but MAbs (0% inhibition) and wells with no serum and no MAbs (100% inhibition), the missing components being replaced with blocking buffer.

1.7 Quantification of C and B-ELISA results and statistical analysis: The inhibition of MAb binding to the specific epitope on the envelope protein in presence of serum was expressed as percent inhibition (PI), calculated from mean OD values using following formula

$$P I = ( 100 - ( \frac{OD test sample}{OD average MAb} ) \times 100 )$$

The mean and standard deviation of percent inhibition values from dengue negative and positive serum samples were calculated and positive cutoff was established. The relative specificity and sensitivity of C-ELISA were estimated by using the VNT as gold standard test.

### 2. Results

2.1 Optimal dilutions of Antigen and MAbs: Since one of the requirements of C-ELISA was the possibility to use non-purified and non-concentrated dengue lysate antigens, the strategy adopted was the use of a capture anti-dengue IgA that was previously captured by anti-human IgA to maxi-sorb ELISA plate. The technique is able to immune-purify and concentrate the dengue viral antigen from cell lysate and to present as trapped antigen to a dengue specific IgG (either from positive serum or specific MAbs). We observed that anti-dengue IgA specific captured antigen purification followed by the detection using dengue specific MAbs (either complex or serotypes specific) picked up more antigens than MAbs captured ELISA (FIG. 1a and 1b).

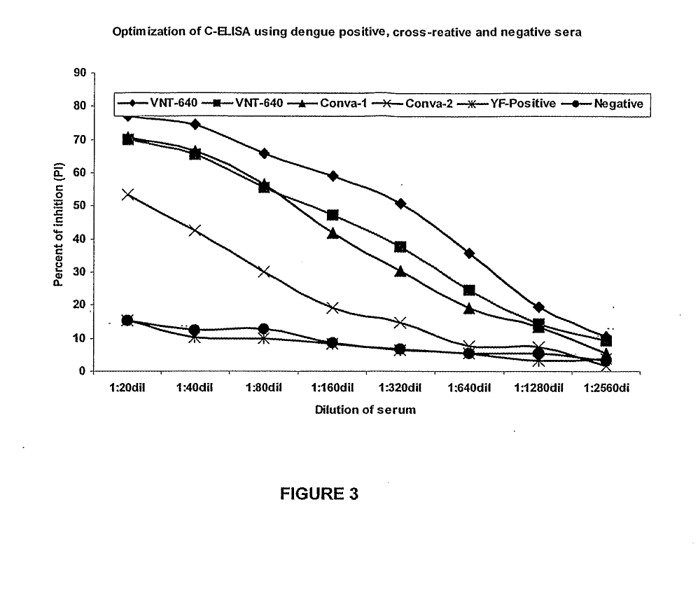

2.2 Optimal dilutions of Antigen, MAbs and serum: The main objective of C-ELISA was to differentiate dengue specific antibody from other members of the family flaviviridae. Consequently, the specificity of the test was measured by its ability to discriminate between weak-positive (as defined by VNT) dengue virus serum and strong positive flavivirus serum (yellow fever) at various concentrations. We expressed specificity as the mean difference in PI values between weak positive dengue serum and strong positive yellow fever serum. FIG. 2a present titration curves of the pan-dengue specific MAbs (Pan-dengue MAb-Abcam, UK and Pan-dengue MAb-ICL, USA) and the dilutions at which they were further tested. FIG. 2b expressed the differentials as a function of serum dilutions. On the basis of the result in FIG. 2b, we adopted the 75 to 80% saturation dilutions (1:1000) as optimal dilution of the MAbs in subsequent testing. At the concentration of MAbs, the optimal dilution of serum was the dilution yielding the peak value. This serum dilution was 1:10 (FIG. 3). It is remarkable that the two MAbs plateau at the same dilution with serum (FIG. 2a). In subsequent testing, we observed that they gave similar results in very test, and we chose to use Pan-dengue MAb, ICL, USA because of its cheaper value and availability.

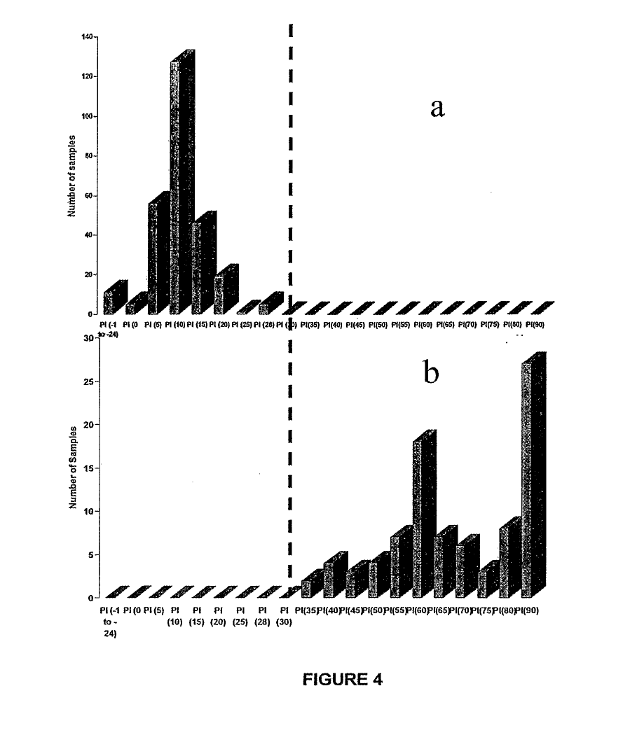

2.3 Test Format: competitive ELISA versus B-ELISA and incubation periods: We compared competitive and blocking ELISA by strong, moderate, weak and negative dengue sera (VNT=1:640, 1:160, 1:40 and <1:10). This comparison was extended by evaluating the values for different combinations of serum and MAbs incubation periods, with sera with optimal dilution. Either MAbs or serum was added simultaneously or MAb added after pre-incubation with serum for total incubation periods (serum plus MAb) of 45, 60, 90 and 120 minutes. FIG. 4 clearly indicates that the competitive and blocking format yield similar sensitivity and specificity. Furthermore, a 30 min pre-incubation with the serum and further 30 min incubation after adding MAb resulted in optimal specificity and sensitivity than simultaneous addition of MAb and serum.

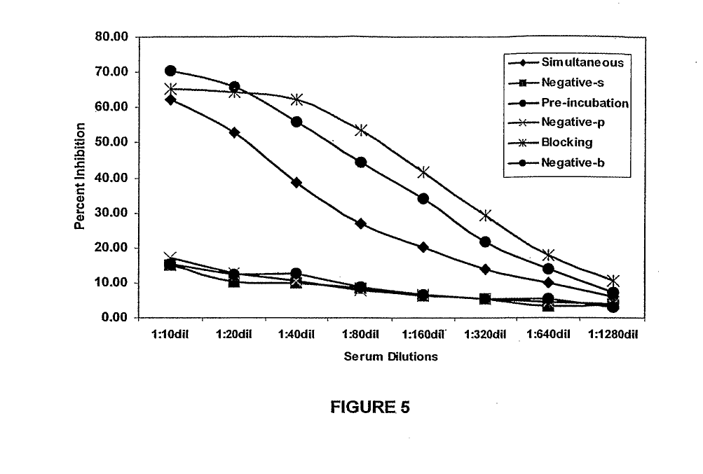

2.4 Negative cutoff value: By using 100 serum samples negative for dengue antibodies (IgG and IgM) using IgM capture ELISA and indirect dengue IgG (Pan-Bio, Australia and IVD Research Inc, Carlsbad, Calif. 92008, USA) and 64 dengue positive sera (VNT>1:20) and the 25 dengue IgG reactive but negative to dengue virus VNT (<1:10), a cutoff value was established for C and B ELISA (FIG. 5). For the two populations combined (FIG. 5a, 5b), the cutoff value was arbitrarily set at the mean (9.9) plus 2 standard deviation or inhibition (6.6). When the flavivirus cross-reactive serum samples are considered alone (FIG. 5, the same cutoff point is given by the mean plus 3 standard deviations. For the absolute negative serum samples, the mean plus standard deviations gave a cutoff value of 25% inhibition.

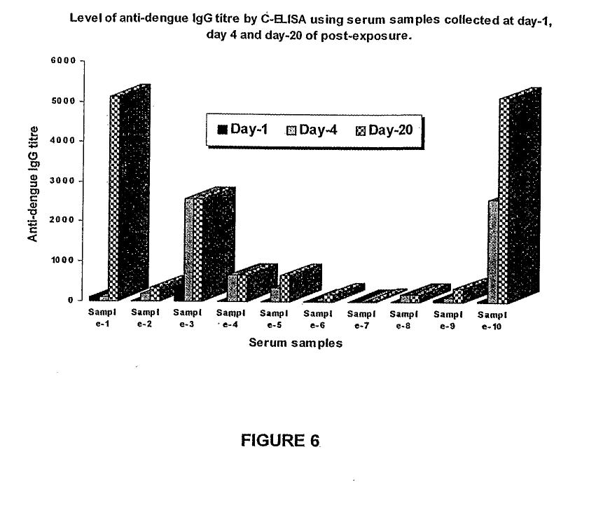

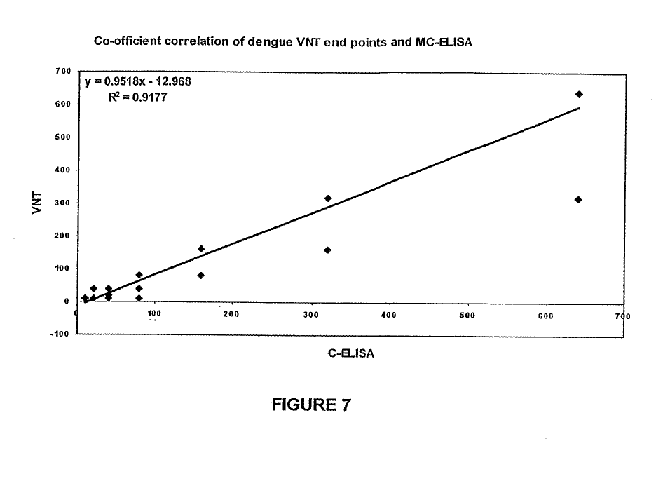

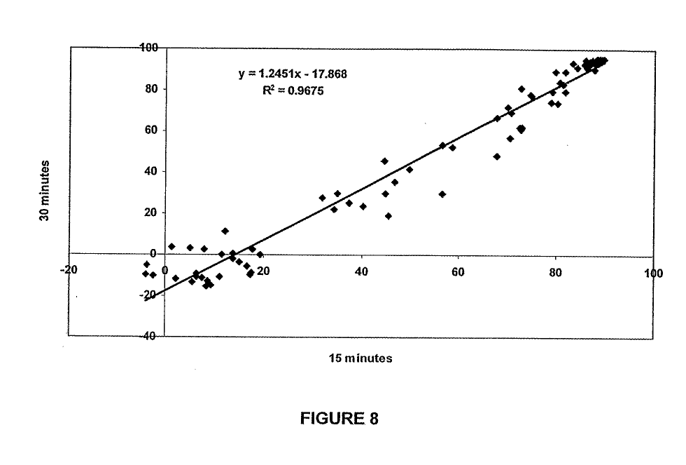

2.5 Comparison of the VNT and C-ELISA: FIG. 6 presents mean endpoint titers for the two sets of serum samples, as determined by the VNT and C-ELISA. The overall correlation coefficients between results of the test was (n=64). By combining all the samples, the specificity and sensitivity of C-ELISA relative to the VNT were estimated as shown Table 1.

Table 1 shows comparison of C-ELISA with virus neutralization test (VNT) for detecting dengue specific antibody (IgG)

TABLE 1
Virus Neutralization Test (VNT)

| | | Positive | Negative | Total |
|---|---|---|---|---|
| C-ELISA | Positive | 62 | 0 | 62 |
| | Negative | 2 | 100 | 102 |
| | Total | 64 | 100 | 164 |

Sensitivity=62/64=1×100=96.88%, Specificity=100/100=1×100=100%, positive predictive value=62/62*100=100 and negative predictive value=100/102*100=98%.

2.6 C-ELISA and Dengue secondary infection: The sensitivity and specificity of C-ELISA for the detecting of dengue secondary infection were compared with two conventional techniques; —anti-dengue IgM and IgG ratio. ≦1.2 (secondary infection) and <1.2 (primary infection, Shu et al., 2003) and anti-dengue IgG capture ELISA, which is equivalent to ≧1:2560 HI unit (Table 3).

Table 2 shows comparison of C-ELISA with conventional techniques (IgM and IgG ratio) of detecting dengue secondary infection.

Table 3 shows comparison of C-ELISA with conventional techniques (dengue capture IgG ELISA) of detecting dengue secondary infection.

TABLE 2
Anti-dengue IgM/IgG ratio (≦1:2 and <1:2)

| | | Positive | Negative | Total |
|---|---|---|---|---|
| C-ELISA | Positive | 64 | 8 | 72 |
| | Negative | 51 | 31 | 82 |
| | Total | 115 | 39 | 154 |

Sensitivity=72/115×100=62.60%, Specificity=31/39×100=79.48%, Positive predictive value (PPV)=64/72×100=88.89% and Negative predictive value=31/82×100=37.80%

TABLE 3
Anti-dengue IgG Capture ELISA

| | | Positive | Negative | Total |
|---|---|---|---|---|
| C-ELISA | Positive | 54 | 18 | 72 |
| | Negative | 16 | 66 | 82 |
| | Total | 70 | 84 | 154 |

Sensitivity=54/70×100=77.14%, Specificity=82/84×100=97.62%, positive predictive value=54/72×100=75% and negative predictive value=66/82×100=80.49%.

2.7. C-ELISA and dengue serotyping: The test was used also to differentiate the serotypes of dengue virus presence in serum samples using dengue serotype specific monoclonal antibodies; Den-1, Den-2, Den-3 and Den-4. Eighty-one dengue specific serum samples previously detected by pan-dengue monoclonal antibodies were further analyzed using dengue serotype specific monoclonal antibodies. Three types of dengue serotype specific Mabs; dengue-2, dengue-4 and dengue-1 were used. Results in Table-4 show that 80.25% were positive to Den-2, 49.38% dengue-4 and 32.10% to dengue-1 virus respectively. Twenty percent sera were positive to all three dengue serotypes (Den-2, Den-4 and Den-1), 38.46% samples were positive to dengue 2 and dengue 4 antibodies, 10.77% sera had antibodies against dengue 2 and dengue-1 while 12.50% serum samples were positive to dengue-4 and dengue 1 viruses. Serum samples were tested against dengue serotype 3 due to the low reactivity of serotypes specific MAb.

TABLE 4
Dengue Serotyping

| | | | | | Den-1 + | | | |
|---|---|---|---|---|---|---|---|---|
| Total | Den- | Den-1 | Den-2 | Den-4 | Den2 + | Den-1 + | Den-1 + | Den-2 + |
| samples | specific | specific | specific | specific | Den-4 | Den-2 | Den-4 | Den-4 |
| 88 | 81 | 26 | 65 | 46 | 13 | 20 | 18 | 25 |
| | 90.05% | 32.10% | 80.25% | 49.38% | 20% | 30.77% | 27.69% | 38.46% |

2.8. Fifteen minutes and one-step competitive ELISA: The test was performed as described in section 2.3 except the competing monoclonal antibody was conjugated with HRP (ICL, USA) and added simultaneously with test serum. The total incubation period was shortened from 90 minutes to 15 minutes and two steps to one step. The result of the 15 minutes, single step assay was compared with pre-incubation C-ELISA and simultaneous 30 minute one step C-ELISA and was found 100% similar (Table-5).

TABLE 5
Comparison of Performance Times

| | | C-ELISA/90 | C-ELISA/30 | C-ELISA/15 |
|---|---|---|---|---|
| | Total | minutes and | minutes and | minutes and |
| | samples | 2 steps | 1 step | I step |
| Positive | 98 | 80 (81.63%) | 83 (84.69%) | 81 (82.65%) |
| (Pan-bio IgG) | | | | |
| Negative | 29 | 1 (3.45%) | 3 (10.35%) | 1 (3.45%) |
| (Pan-bio IgG) | | | | |

2.9 Competitive ELISA against Japanese Encephalitis Virus: Monoclonal-based C-ELISA was also established against non-dengue flaviviruses like Japanese encephalitis virus (JEV) using virus specific monoclonal antibody (cross-reactive with St. Louis Encephalitis Virus). The plate was prepared using the protocol described in section 2.3 and JEV antigen was captured instead of dengue virus. In short, JE lysate antigen (Nayakama strain) was used and captured by anti-dengue IgA. Anti-JE antibody was produced in rabbit and used for the development of the assay and 83 human serum samples were screened using the technique. Of 83 samples 65 samples were positive to flavivirus cross-reactive IgG and the reminder 28 sera negative to flaviviruses. Results showed that 89.23% (58/65) flavivirus reactive sera were positive by JE C-ELISA while 89.29% (25/28) flavivirus negative samples did not react with JE-C-ELISA. However, validation of the technique was not done using JE confirmed serum samples.

### 3. Discussion

The MAb-based competitive ELISA proved to be a rapid, sensitive and specific method for detection of dengue antibody. The test offers the additional advantage, over indirect ELISA, of permitting screening of sera from different members of flavivirus. Compared to VNT, C-ELISA offers high levels of sensitivity (100%) and specificity (100%) while decreasing the rum time from 7 days to less than 2 hours. Furthermore, unlike the VNT, C-ELISA may less affect by the quality of sera (cytotoxicity, contamination, hemolyzed or rich in fat) and low level of cross-reactivity against other members of flavivirus. Contaminations may affect the outcome of serology in two ways: degradation of antibody and alteration of pH to a level that hinders antibody binding. In practice, the relatively high dilution at which serum is screened (1:20 or above) should nullify the second factor.

The superiority of B-ELISA and 30 minutes pre-incubation serum C-ELISA over simultaneous addition of test serum and MAb may be due to the fact that MAb is highly purified and has a high-affinity for the epitope than serum antibodies do. When the two are added simultaneously, only high-affinity serum antibody would compete in a competitive format, early-primary-response sera, which would contain mainly low-affinity antibody, tested negative while secondary-response sera, presumably having high affinity, showed a level of sensitivity comparable to that of B-ELISA. However the use of conjugated monoclonal antibody specific to dengue virus has eliminated the lower sensitivity of the technique and similar results were obtained. The modified technique not only improved the sensitivity of the test but also reduced the total timing from 90 minutes to 15 minutes and two steps to one step. The 15 minute, one step C-ELISA makes a tremendous break-through of the current dengue IgG detection technique and a single test can be used to differentiate between dengue primary and secondary infection at the early stage of disease as well as dengue specific sero-surveillance which is particularly important where there is more than one flavivirus co-circulating. Currently two types of anti-dengue IgG test are used for the detection of dengue secondary infection and sero-surveillance.

The approach of standardization of the C-ELISA to a micro-neutralization assay for two reasons: (i) the VNT is presently the only available confirmatory test of dengue infection where more than one flavivirus is present and (ii) neutralizing antibodies are considered the best predictor of host immune status. The C-ELISA results agreed very well with those of the VNT and there was a very high positive correlation coefficient (r=0.88) between two test results.

Detection of dengue specific antibody is not only important for epidemiological study but also to diagnose secondary infection through the detection of dengue specific IgG which patient might carry from the previous infection. According to WHO, only serum having >2560 HAI level of anti-dengue IgG in the early convalescence phase of dengue infection is considered as secondary infection (WHO Manual-1982). However, this criterion of detection of dengue secondary infection may lead the patient to undergo severe form of dengue infection like dengue hemorrhagic fever (DHF) because of delaying detection procedure and thus not applicable to take proper patient management. We compare our new technique with a WHO equivalent test (dengue IgG capture ELISA, Table 3) and found that C-ELISA is 77% sensitive and 97.62% specific to dengue IgG capture ELISA and it was also observed that 18 dengue cases despite the presence of dengue specific IgG, capture IgG failed to detect, while 16 dengue cases were detected by capture IgG ELISA which were not specific to dengue IgG. Hence, the presence of high-level dengue cross-reactive IgG does not mean the dengue secondary infection, which is a common scenario where more than one flavivirus is co-circulating. On the other hand, presence of low level of dengue specific IgG in an early phase of dengue illness does not rule out the dengue secondary cases. Another technique described by Shu et al., 2003 using IgM and IgG ratio (Table 2) showed 74.67% dengue patients had a secondary infection, which is pretty high compared to dengue capture IgG and C-ELISA. Under this circumstance, we found that our invented technique C-ELISA is a powerful tool to detect secondary infection by detecting dengue specific IgG (both at low and high levels) even within 15 minutes that presence in the patient serum during dengue acute illness.

The protocol developed for C-ELISA was demonstrated using dengue lysate antigen and dengue specific monoclonal antibody. This can also be used against other flaviviruses and the preliminary results in Japanese encephalitis is an example of this.

Finally it is to be understood that various other modifications and/or alterations may be made without departing from the spirit of the present invention as outlined herein.

### REFERENCES

Gubler, D. J., Sather, G. E. (1988). Laboratory diagnosis of dengue and dengue hemorrhagic fever. In A. Homma, and J. F. Cunha (ed.), Proceedings of the International Symposium on Yellow Fever and Dengue. p. 291-322.
Gubler, D. J. 1996. Serologic diagnosis of dengue/dengue haemorrhagic fever. Dengue Bull. 20:20-23. Gubler, D. J. (1998). Dengue and dengue hemorrhagic fever. Clin Microbiol Rev. 11 (3):480-96.
Gubler, D. J. (2002). Epidemic dengue/dengue hemorrhagic fever as a public health, social and economic problem in the 21st century. Trends Microbiol. 10(2):100-3. Innis, B. L., A. Nisalak, S, Nimmannitya, S. Kusalerdchariya, V. Chongswasdi, S. Suntayakorn, P. Puttisri, and C. H. Hoke. 1989. An enzyme-linked immunosorbent assay to characterize dengue infections where dengue and Japanese encephalitis co-circulate. Am. J. Trop. Med. Hyg. 40:418-427.
Shu, P. Y., L. K. Chen, S. F. Chang, Y. Y. Yueh, L. Chow, L. J. Chien, C. Chin, T. H. Lin, and J. H. Huang. 2003. Comparison of capture immunoglobulin M (IgM) and IgG enzyme-linked immunosorbent assay (ELISA) and nonstructural protein NS1 serotype-specific IgG ELISA for differentiation of primary and secondary dengue virus infections. Clin. Diagn. Lab. Immunol. 10:622-630.
World Health Organization. 1997. Dengue haemorrhagic fever: diagnosis, treatment, prevention and control, 2nd ed. World Health Organization, Geneva, Switzerland.
WHO: Fact sheet No: 117. (2002). Dengue and Dengue Haemorrhagic fever. http://www.who.int/inf-fs/en/fact117.html
Ooi, E. E., Hart, T. J., Tan, H. C., Chan, S. H. (2001). Dengue seroepidemiology in Singapore. Lancet. 357(9257):685-6.
Simmons, M., Jennings, G., Oprandy, J. J. (1993). A rapid membrane based immunobinding assay for the detection of dengue virus in tissue culture. Southeast Asian J Trop Med Public Health. 24(4):742-6.
Wu, S. J., Paxton, H., Hanson, B., Kung, C. G., Chen, T. B., Rossi, C., Vaughn, D. W., Murphy, G. S., Hayes, C. G. (2000). Comparison of two rapid diagnostic assays for detection of immunoglobulin M antibodies to dengue virus. Clin Diagn Lab Immunol. 7(1):106-10.
Stollar, V., Stevens, T. M., Schlesinger, R. W. (1966). Studies on the nature of dengue viruses. II. Characterization of viral RNA and effects of inhibitors of RNA synthesis. Virology. 30(2):303-12. Newton, E. A., Reiter, P. (1992). A model of the transmission of dengue fever with an evaluation of the impact of ultra-low volume (ULV) insecticide applications on dengue epidemics. Am J Trop Med Hyg. 47(6):709-20.
Platt, K. B., Linthicum, K. J., Myint, K. S., Innis, B. L., Lerdthusnee, K., Vaughn, D. W. (1997). Impact of dengue virus infection on feeding behavior of Aedes aegypti. Am J Trop Med. Hyg. 57(2):119-25.
Scott, T. W., Naksathit, A., Day, J. F., Kittayapong, P., Edman, J. D. (1997). A fitness advantage for Aedes aegypti and the viruses it transmits when females feed only on human blood. Am J Trop Med. Hyg. 57(2):235-9.
Nawa, M., Takasaki, T., Yamada, K. I., Akatsuka, T., Kurane, I. (2001). Development of dengue IgM-capture enzyme-linked immunosorbent assay with higher sensitivity using monoclonal detection antibody. J Virol Methods. 92(1):65-70.
Groen, J., Koraka, P., Velzing, J., Copra, C., Osterhaus, A. D. (2000). Evaluation of six immunoassays for detection of dengue virus-specific immunoglobulin M and G antibodies. Clin Diagn Lab Immunol 7(6):867-71.
Halstead, S. B. (1974). Etiologies of the experimental dengues of Siler and Simmons. Am J Trop Med Hyg. 23(5):974-82.

1. A method for detecting exposure of a subject to a flavivirus, said method comprising the steps of:
contacting a biological sample from the subject with a mixture of anti-flavivirus IgA captured components of flavivirus;
subjecting the biological sample and the anti-flavivirus IgA captured components to a competing flavivirus specific monoclonal antibody; and
determining the presence of a complex that forms between a flavivirus IgG specific binding partner present in the biological sample and the anti-flavivirus IgA captured component of flavivirus in the presence of the competing flavivirus specific monoclonal antibody.

2. The method according to claim 1 wherein the biological sample and the anti-flavivirus IgA captured components of flavivirus are pre-incubated to form a complex prior to subjecting to the competing flavivirus specific monoclonal body.

3. The method according to claim 1 wherein the biological sample, the anti-flavivirus IgA captured component of flavivirus and the competing flavivirus specific monoclonal antibody are contacted simultaneously prior to determining the presence of a complex.

4. The method according to claim 1 wherein the competing flavivirus specific immunological agent is an antibody specific to an epitope of an anti-flavivirus IgA captured component of flavivirus.

5. The method according to claim 1 wherein the competing flavivirus specific monoclonal antibody is conjugated to a reporter group.

6. (canceled)

7. The method according to claim 1 wherein the anti-flavivirus IgA captured components are captured from a lysate of cells infected with flavivirus.

8. The method according to claim 1 wherein the anti-flavivirus IgA captured component of flavivirus is selected from the group consisting of flavivirus structural and non-structural proteins, flavivirus particles and fragments thereof, glycoproteins, lipids and carbohydrates derived from the flavivirus.

9. The method according to claim 8 wherein the structural protein is selected from the group consisting of envelope proteins, Pr membrane proteins, and nucleocapsid proteins.

10. The method according to claim 9 wherein the anti-flavivirus IgA captured component is an anti-idiotypic antibody to an antigen binding site of a flavivirus antibody generated in response to exposure to an IgA captured component derived from a flavivirus or equivalent thereof.

11. The method according to claim 1 wherein the binding partner is an antibody expressed in an early stage of a flavivirus infection, during convalescence or derived from a previous infection.

12. The method according to claim 1 wherein the flavivirus is selected from the group consisting of yellow fever virus, dengue virus, and JE virus.

13. (canceled)

14. (canceled)

15. The method according to claim 12 wherein the method detects exposure to a dengue virus serotype selected from the group consisting of DEN-1, DEN-2, DEN-3 and DEN-4.

16. The method according to claim 15 wherein the competing dengue specific immunological agent is an antibody specific to an epitope common to dengue virus serotypes DEN-1, DEN-2, DEN-3 or DEN-4.

17. The method according to claim 12 comprising a non-structural protein selected from the group consisting of NS-1, NS-2a, NS-2b, NS-3, NS-4a, NS-4b and NS-5.

18. (canceled)

19. The method according to claim 12 wherein the flavivirus IgG specific binding partner is an IgG antibody that is specific to a dengue serotype selected from the group consisting of DEN-1, DEN-2, DEN-3 and of DEN-4.

20. The method according to claim 1 wherein the biological sample is selected from the group consisting of blood, saliva, cord fluid, B cells, T cells, plasma, serum, urine and amniotic fluid.

21. (canceled)

22. A mixture of anti-flavivirus IgA captured components of flavivirus.

23. The mixture of anti-flavivirus IgA captured components according to claim 22 captured from a lysate of cells infected with flavivirus.

24. The mixture according to claim 22 comprising components selected from the group consisting of flavivirus structural and non-structural proteins, flavivirus particles and fragments thereof, glycoproteins, lipids and carbohydrates derived from the flavivirus.

25. The mixture according to claim 24 wherein the structural protein is selected from the group consisting of envelope proteins, Pr membrane proteins, and nucleocapsid proteins.

26. The mixture according to claim 22 wherein the flavivirus is selected from the group consisting of yellow fever virus, dengue virus, and JE virus.

27. (canceled)

28. (canceled)

29. The mixture according to claim 26 wherein the dengue virus is selected from the group consisting of DEN-1, DEN-2, DEN-3 and DEN-4.

30. The mixture according to claim 26 comprising a non-structural protein selected from the group consisting of NS-1, NS-2a, NS-2b, NS-3, NS-4a, NS-4b and NS-5.

31. (canceled)

32. A kit for detecting exposure of a subject to a flavivirus, said kit comprising;
anti-flavivirus IgA captured components from flavivirus;
a competing flavivirus specific monoclonal antibody; and
at least one detection agent for detecting a complex that forms between a IgG specific binding partner present in a biological sample and an anti-flavivirus IgA captured component in the presence of the competing flavivirus specific monoclonal antibody.

33. The kit according to claim 32 wherein the anti-flavivirus IgA captured component or the competing flavivirus specific monoclonal antibody is immobilized on a solid support.

34. The kit according to claim 32 wherein the anti-dengue IgA captured component is selected from the group consisting of flavivirus virus structural and non-structural proteins, flavivirus particles and fragments thereof, glycoproteins, lipids and carbohydrates derived from the flavivirus.

35. The kit according to claim 34 wherein the structural protein is selected from the group consisting of envelope proteins, Pr membrane proteins, and nucleocapsid proteins.

36. The kit according to claim 32 wherein the flavivirus is selected from the group consisting of yellow fever virus, dengue virus, and JE virus.

37. (canceled)

38. (canceled)

39. The kit according to claim 36 wherein the competing flavivirus specific monoclonal antibody is a dengue specific antibody specific to an epitope common to dengue virus serotypes DEN-1, DEN-2, DEN-3 or DEN-4.

40. The kit according to claim 36 comprising a non-structural protein selected from the group consisting of NS-1, NS-2a, NS-2b, NS-3, NS-4a, NS-4b and NS-5.

41. (canceled)

42. A solid support
comprising anti-flavivirus IgA captured components immobilized on the support.

43. The solid support according to claim 42, wherein the support is selected from the group consisting of a bead, a disc, a magnetic particle or a fiber optic sensor, a microtitre plate, glass slide or biological microchip or a membrane including nitrocellulose membranes, polytetrafluorethylene membrane filters, cellulose acetate membrane filters and cellulose nitrate membrane filters with filter paper carriers.

44. A method of assessing the relative risk of one or more subjects being exposed to flavivirus within a defined location comprising;
a) obtaining samples from a representative population within a defined location; and
b) assessing exposure of individual members of a sample population to a flavivirus by the method comprising the steps of:
i) contacting a biological sample from a subject with an anti-flavivirus IgA captured component derived from the flavivirus to form a complex between the component and a flavivirus specific binding partner present in the biological sample;
ii) determining the presence of the complex that forms between a flavivirus specific binding partner present in the biological sample and an anti-flavivirus IgA captured component in the presence of a competing flavivirus specific immunological agent, wherein the presence of the complex is indicative of exposure of the subject to a flavivirus; and
c) assessing the relative risk of exposure within the defined location.
